(19) United States
(12) Patent Application Publication
(10) Pub. No.: US 2021/0369828 A1
(43) Pub. Date: Dec. 2, 2021

(54) PLANT VIRUS BASED CANCER ANTIGEN VACCINE

(71) Applicant: CASE WESTERN RESERVE UNIVERSITY, Cleveland, OH (US)

(72) Inventors: Nicole F. Steinmetz, San Diego, CA (US); Sourabh Shulka, Cleveland, OH (US)

(21) Appl. No.: 17/333,797

(22) Filed: May 28, 2021

Related U.S. Application Data
(60) Provisional application No. 63/031,376, filed on May 28, 2020.

(51) Int. Cl. A61K 39/00; C12N 7/00; A61P 35/00

(52) U.S. Cl. CPC A61K 39/001188; A61K 2039/5258; A61P 35/00; C12N 7/00

(57) ABSTRACT

A vaccine composition includes an icosahedral-shaped plant virus or virus-like particle linked to a plurality of NY-ESO-1 antigens.

### RELATED APPLICATION

This application claims priority from U.S. Provisional Application No. 63/031,376, filed May 28, 2020, the subject matter of which is incorporated herein by reference in its entirety.

### GOVERNMENT FUNDING

This invention was made with government support under grant CA224605 awarded by the National Institutes of Health. The government has certain rights in the invention.

### BACKGROUND

One of the most sought-after areas in vaccine development is the development of cancer immunotherapies. Peptide-based vaccines constitute the largest group of cancer vaccines under preclinical and clinical evaluation. Cancer vaccines targeted to tumor-associated antigens (TAAs) can improve disease-free survival through immune system-mediated elimination of residual or recurring disease. Nevertheless, peptide-based vaccines suffer from weak and short-lived immunogenicity and are dependent on adjuvants. In the absence of suitable adjuvants the peptides are prone to proteolytic degradation resulting in shorter circulation times. Thus, there is a need for improved vectors and epitope presentation strategies to develop stable peptide-based vaccines.

Aberrant expression of the cancer testis antigen NY-ESO-1 has been found in several malignancies including triple negative breast cancer, melanomas, myelomas and ovarian cancer. NY-ESO-1⁺ malignancies are characterized by the presence of low levels of spontaneous antigen-specific humoral and cellular immune responses, including elevated CD8⁺ T cell infiltration and IFN-γ levels. However, these spontaneous responses are highly abated by the immunosuppressive tumor microenvironment, which prevents immunological clearance of cancer. Therefore, cancer immunotherapies provide an opportunity to initiate or amplify pre-existing anti-tumor immunities to clear primary tumors and metastasis as well as prime immune memory against recurring cancers.

Currently, several NY-ESO-1 vaccines are under evaluation including dendritic cell (DC)-based vaccines, recombinant proteins or peptide subunit vaccines, and mammalian viral vectors. These approaches are hampered by numerous limitations: DC-based vaccines need to be custom-made for each patient and can be technically challenging and expensive; recombinant proteins could be costly to synthesize and suffer from inefficient or non-specific responses arising due to misfolded proteins and inefficient presentation of epitopes. Subunit peptide vaccines require adjuvants for enhanced immunogenicity and stability, many of which raise serious safety concerns. Mammalian viral vector expression systems carry antigen-coding RNA and depend on protein expression in targeted cells, accompanied by proteolytic processing and presentation of appropriate antigenic peptides to stimulate a cellular immune response.

### SUMMARY

Embodiments described herein relate to a vaccine composition and, particularly, relates to a plant virus based cancer antigen vaccine. The vaccine composition includes a plurality of icosahedral-shaped plant viruses or plant virus-like particles linked to a plurality of NY-ESO-1 antigens. The plurality of NY-ESO-1 antigens can be conjugated to the external surface of the plant viruses or plant virus-like particles. The vaccine composition can further include a pharmaceutically acceptable carrier and/or an adjuvant.

In some embodiments, the plant virus or plant virus-like particle is of the Secoaviridae family. In some embodiments, the plant virus or plant virus-like particle is of the genus Comovirus, such as a cowpea mosaic virus (CPMV) or CPMV virus-like particle.

In some embodiments, the NY-ESO-1 antigen includes all or a portion of the amino acid sequence located between position 155 and 167 of the NY-ESO-1 protein. In some embodiments, the NY-ESO-1 antigen includes a peptide having the amino acid sequence selected from SLLMWITQCFL (SEQ ID NO:2), SLLMWITQC (SEQ ID NO:3), and QLSLLMWIT (SEQ ID NO:4). In an exemplary embodiment, the NY-ESO-1 antigen includes a peptide having the amino acid sequence SLLMWITQV (SEQ ID NO:1). In some embodiments, the NY-ESO-1 antigen includes a cysteine terminated NY-ESO-1 peptide with an intervening flexible linker, such as a peptide having the amino acid sequence SLLMWITQV-LSPG-C(SEQ ID NO:5).

Additional embodiments described herein relate to methods of treating or decreasing the risk of developing a NY-ESO-1-expressing cancer in a subject. The method includes administering to a subject in need thereof an effective amount of a vaccine composition that includes an icosahedral-shaped plant virus or virus-like particle linked to a plurality of NY-ESO-1 antigens. The plurality of NY-ESO-1 peptides can be conjugated to the external surface of the plant virus or plant virus-like particle. The vaccine composition can further include a pharmaceutically acceptable carrier. In some embodiments, the therapeutically effective amount of the vaccine composition administered to the subject for the treatment of cancer is an amount effective to enhance uptake and activation of antigen presenting cells and promote a potent CD8+ T cell response in the subject.

In some embodiments, the icosahedral-shaped plant virus or plant virus-like particle is of the Secoaviridae family. In some embodiments, the icosahedral-shaped plant virus or plant virus-like particle is of the genus Comovirus, such as a cowpea mosaic virus (CPMV) or CPMV virus-like particle.

In some embodiments, the NY-ESO-1 antigen includes all or a portion of the amino acid sequence located from position 155 and 167 of the NY-ESO-1 protein. In some embodiments, the NY-ESO-1 antigen includes a peptide having the amino acid sequence selected from SLLMWITQCFL (SEQ ID NO:2), SLLMWITQC (SEQ ID NO:3), and QLSLLMWIT (SEQ ID NO:4). In some embodiments, the NY-ESO-1 antigen includes a peptide having the amino acid sequence SLLMWITQV (SEQ ID NO:1). In some embodiments, the NY-ESO-1 antigen includes a cysteine terminated NY-ESO-1 peptide with an intervening flexible linker, such as a peptide having the amino acid sequence SLLMWITQV-LSPG-C(SEQ ID NO:5).

In some embodiments, the NY-ESO-1-expressing cancer is selected from triple negative breast cancer, melanoma, myeloma and ovarian cancer. In some embodiments, the NY-ESO-1-expressing cancer is a melanoma.

Vaccine compositions described herein can be administered parenterally. In some embodiments, the vaccine composition is administered subcutaneously.

In some embodiments, the method of treating or decreasing the risk of developing a NY-ESO-1-expressing cancer in a subject can further include administering a therapeutically effective amount an additional anticancer agent or therapy to the subject. In some embodiments, the additional anticancer agent is an antitumor agent, ablation and/or radiation therapy. In some embodiments, the method further includes administering an adjuvant to the subject.

### BRIEF DESCRIPTION OF THE FIGURES

FIGS. 1(A-D) illustrate schematics, an immunoblot, and image showing synthesis and characterization of the CPMV-NY-ESO-1 nanoparticles. (A) Structure of CPMV created with UCSF Chimera (v1.12) using the Protein Data Bank entry 1NY7 shows icosahedral morphology. The reactive Lysine residues on the capsid and the asymmetric unit are highlighted in green. (B) NY-ESO-1 peptide with the flexible LSPG linker with a C-terminal Cys was conjugated through the solvent-exposed Lys residues using NHS-chemistry via the bifunctional linker SM (PEG)12. (C) Denaturing 4-12% Nu-PAGE gel stained with GelCode Blue Safe protein stain was used to confirm the successful conjugation of the peptide to the CPMV coat proteins (SCP and L-CP). (D) Transmission electron microscopic (TEM) images of the negatively stained CPMV-NY-ESO-1 nanoparticles show particle integrity post-conjugation.

FIGS. 2(A-C) illustrate images and graphs showing, CPMV-NY-ESO-1 is taken up by the antigen-presenting cells and triggers activation. (A, B) Confocal microscopy was used to verify the uptake of fluorescent CPMV-NY-ESO-1TMR vaccine particles and soluble NY-ESO-1TMR peptides by murine macrophage RAW 264.7 cells. Cell nuclei were stained with 4′,6-diamidino-2-phenylindole (DAPI), the cell membrane was stained with WGA-A555 (red), and fluorescent TMR was used for monitoring the peptide/CPMV-peptide. (C) Immunostimulation of bone marrow-derived dendritic cells (BMDCs) derived from the transgenic HLA-A2 mice by the CPMV-NY-ESO-1 vaccine was examined by incubating 500 000 BMDCs with the CPMV-NYESO-1 vaccine and the soluble NY-ESO-1 peptide for 24 h and quantifying the cytokines (ILβ1, TNF-α, IL-6, and IL-12p70) released in the culture supernatant using enzyme-linked immunosorbent assays (ELISAs). The results were compared using one-way analysis of variance (ANOVA) (with ****=p<0.0001).

FIGS. 3(A-E) illustrate schematics and graphs showing immunogenicity of the CPMV-NY-ESO-1 vaccine. (A) Transgenic HLA-A2 mice (n=5) were immunized subcutaneously 2× biweekly with 50 μg of the CPMV-NY-ESO-1 vaccine or equivalent amount of NY-ESO-1 peptide mixed with the CFA adjuvant (CFA+NY-ESO-1); the IFA adjuvant was used for the booster dose on day 14. Spleens were harvested from immunized mice 2 weeks following the booster dose and CD8+ T cells were isolated. (B) Schematic representation of assays used to determine antigen-specific CD8+ T cell proliferation. (C, D) T cell proliferation was measured using 3-(4,5-dimethylthiazol-2-yl)-2,5-diphenyltetrazolium bromide (MTT) assays (C) and quantification of secreted IFN-γ (D) using ELISAs to determine epitope-specific immunostimulation. CD8+ T cells harvested from immunized mice were co-cultured with NY-ESO-1 peptide-pulsed BMDCs from naive HLA-A2 mice. Nonpulsed BMDCs and NY-ESO-1 peptide (without BMDCs) were used as controls. (E) Antigen specificity of T cell response was determined by co-culturing CD8+ T cells from CPMV-NY-ESO-1-immunized mice with BMDCs pulsed with NY-ESO-1 peptide or irrelevant HER2 peptide P4 and comparing IFN-γ secretion. The results were compared using one-way ANOVA (with ****=p<0.0001).

FIGS. 4(A-B) illustrates a schematic and plot showing antigen-specific cancer cell lysis. (A) CD8+ T cells from CPMV-NY-ESO-1 and CFA+NY-ESO-1-immunized mice were cultured for 3 days with NY-ESO-1 peptide. Activated CD8+ T cells were then co-cultured at varying effector/target cell ratios with NYESO-1+A375 cancer cells or NY-ESO-1− B16F10-OVA cells. (B) Cytotoxicity of CD8+ T cells was determined using MTT assays. The results were compared using one-way ANOVA (with ****=p<0.0001).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

As used herein, the terms “peptide,” “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “nanoparticle” refers to any particle having a diameter of less than 1000 nanometers (nm). In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

The phrases “parenteral administration” and “administered parenterally” are art-recognized terms and include modes of administration other than enteral and topical administration, such as injections, and include, without limitation, intratumoral, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., tumor site), such that it enters the animal's system and, thus, is subject to metabolism and other like processes, for example, subcutaneous administration.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The term “effective amount” or “therapeutically effective amount” refers to a sufficient amount of the composition used in the practice of the invention that is effective to provide effective treatment in a subject, depending on the compound being used. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “therapeutic” treatment is a treatment administered to a subject who exhibits signs of pathology of a disease or disorder for the purpose of diminishing or eliminating those signs.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder, for example decreasing the risk of developing pathology associated with a NY-ESO-1-expressing cancer. In some embodiments, use of a vaccine composition described herein in a preventive treatment provides immunoprotection.

The term “adjuvant” as used herein, refers to an agent that augments, stimulates, potentiates and/or modulates an immune response in an animal. An adjuvant may or may not have an effect on the immune response in itself. Examples of adjuvants include complete Freund's adjuvant (CFA), muramyl dipeptide, Gerbu, and monophosphoryl lipid A.

The terms “immunogen”, “antigen” and “antigenic peptide (epitope)” as used herein refer to a portion or portions of molecules which are capable of inducing a specific immune response in a subject alone or in combination with an adjuvant. An epitope generally represents a portion of an antigen.

The term “immune response”, as used herein, refers to an alteration in the reactivity of the immune system of an animal in response to an antigen or antigenic material and may involve antibody production, induction of cell-mediated immunity, complement activation, development of immunological tolerance, or a combination thereof.

The term “immunoprotection” as used herein, mean an immune response that is directed against one or more antigen so as to protect against disease and/or infection by a pathogen in a vaccinated animal. For purposes of the present invention, protection against disease includes not only the absolute prevention of the disease, but also any detectable reduction in the degree or rate of disease, or any detectable reduction in the severity of the disease or any symptom in the vaccinated animal as compared to an unvaccinated infected or diseased animal. Immunoprotection can be the result of one or more mechanisms, including humoral and/or cellular immunity.

The term “vaccine”, as used herein, refers to a material capable of producing an immune response after being administered to a subject.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to a vaccine composition and, particularly, relates to a plant virus based cancer testis antigen NY-ESO-1 vaccine and/or adjuvant immunotherapy and its use in treating NY-ESO-1 malignancies, such as NY-ESO-1 expressing cancers. The NY-ESO-1 cancer vaccine can be based on the use of plant viral nanoparticle cowpea mosaic virus (CPMV) as an epitope display platform. The CPMV platform can provide enhanced uptake of NYESO-1 peptides into antigen-presenting cells and lead to improved activation of immune cells. The CPMV-NY-ESO-1 vaccine was found to trigger a potent CD8+ T cell response in transgenic HLA-A2 mice and demonstrated antigen-specific lysis of NYESO-1+ cancer cells. The efficacy of this vaccine can be attributed to the inherent immunogenicity, cellular tropism toward immune cells, and efficient lymphatic trafficking.

A vaccine-based immunotherapy approach could particularly benefit patients with NY-ESO-1+ malignancies, such as triple-negative breast cancer (TNBC) that frequently exhibits local and regional recurrence and metastatic relapse within 5 years following surgical resection of the primary tumor and have no tumor-specific treatment options.

A plant virus-based approach to vaccines offers several advantages. Genetic engineering can be used to express epitopes on the viral capsid leading to a homogenous formulation and mitigate heterogeneity of the chemical conjugation methods. Such genetically engineered vaccines can also be propagated in and purified from host plants using molecular farming, thereby reducing downstream processing and cost. Furthermore, plant virus-based vaccines could also be incorporated into polymeric implants and devices, which improves the shelf life of the product and will enable the extended release of the antigen.

In some embodiments, the vaccine composition can be used for methods of treating or decreasing the risk of developing an NY-ESO-1⁺ cancer in a subject by administering to the subject in need thereof a therapeutically effective amount of the vaccine composition.

The vaccine composition includes an icosahedral plant virus or virus-like particle (VLP) linked to a plurality of NY-ESO-1 antigens. In some embodiments, the NY-ESO-1 antigens can be conjugated to an exterior surface of the icosahedral plant virus particle. The plant virus particles can facilitate efficient delivery of NY-ESO-1 antigens to antigen presenting cells (APCs) in the subject to promote immune system stimulus and the processing and presentation of the antigens. Thus, it is contemplated that plant virus-based NY-ESO-1 vaccine composition can prime an effective anti-NY-ESO-1 CD8+ CTL response, delay tumor progression, and improve survival in a subject with cancer.

The icosahedral-shaped plant virus particles or plant virus-like particles can be nonreplicating and noninfectious in the subject to avoid infection of the subject and can be regarded as safe from a human health and agricultural perspective. In planta production prevents endotoxin contamination that may be a byproduct of other virus or virus-like particle systems derived from E. coli. The plant virus particles or VLPs are scalable, stable over a range of temperatures (4-60° C.) and solvent:buffer mixtures. For example, CPMV can be propagated in and purified from Vigna unguiculata plants with yields of 50-100 mg virus/100 g of infected leaves.

In some embodiments, icosahedral-shaped plant virus particles or plant virus-like particles in which the viral nucleic acid is not present are conjugated to an NY-ESO-1 peptide antigen. Virus-like particles lacking their nucleic acid are non-replicating and non-infectious regardless of the subject into which they are introduced.

In other embodiments, the icosahedral-shaped plant virus particles include a nucleic acid within the virus particle. If present, the nucleic acid will typically be the nucleic acid encoding the virus. However, in some embodiments the viral nucleic acid may have been replaced with exogenous nucleic acid. In some embodiments, the nucleic acid is RNA, while in other embodiments the nucleic acid is DNA. A virus particle including nucleic acid will still be nonreplicating and noninfectious when it is introduced into a subject which it cannot infect. For example, plant virus particles will typically be nonreplicating and noninfectious when introduced into an animal subject.

An icosahedral-shaped plant virus is a virus that primarily infects plants, is non-enveloped and has capsid proteins that can self-assemble into well-organized icosahedral three-dimensional (3D) nanoscale multivalent architectures with high monodispersity and structural symmetry. Icosahedral-shaped plant viruses also include an exterior surface and interfaces between coat protein (CP) subunits that can be manipulated to allow for controlled self-assembly, and multivalent ligand display of nanoparticles or molecules for varied applications.

In some embodiments, the icosahedral plant virus is a plant picornavirus. A plant picornavirus is a virus belonging to the family Secoaviridae, which together with mammalian picornaviruses belong to the order of the Picornavirales. Plant picornaviruses are relatively small having a diameter of about 30 nm, non-enveloped, positive-stranded RNA viruses with an icosahedral capsid. Plant picornaviruses have a number of additional properties that distinguish them from other picornaviruses, and are categorized as a subfamily of Secoviridae. In some embodiments, the plant virus particles are selected from the Comovirinae virus subfamily. Exemplary Comovirinae subfamily viruses for use in a method described herein can include Cowpea mosaic virus (CPMV), Broad bean wilt virus 1, and Tobacco ringspot virus. In certain embodiments, the plant virus or plant virus-like particles are from the genus Comovirus. A preferred example of a Comovirus is the CPMV or CPMV-like virus particles. The immune stimulating ability of CPMV is derived from its highly organized 3D protein architecture with its encapsulated nucleic acid and an intrinsic immune cell tropism. In some embodiments, the plant virus-like particle is an empty cowpea mosaic virus-like particle (eCPMV).

Vaccine compositions described herein also include an NY-ESO-1 antigen. NY-ESO-1 antigens can include portions of the NY-ESO-1 protein, that are recognized by the immune system; e.g., by antibody binding. The 180-amino acid NY-ESO-1 polypeptide is encoded by the CTAG1B gene and is expressed in a variety of malignant human tumors. Structurally, NY-ESO-1 features a glycine-rich N-terminal region, as well as a hydrophobic C-terminal region with a Pcc-1 domain. An advantage of presenting the NY-ESO-1 antigen linked to a plant virus particle is that such linked particles are capable of stimulating an immune response without having to be co-administered with an adjuvant.

It will be appreciated that any NY-ESO-1 antigen capable of eliciting an immune response can be used in the vaccine composition described herein. In some embodiments, the NY-ESO-1 antigen can include a peptide sequence corresponding to an HLA-A2 restricted epitope in NY-ESO-1 identified as a recognition site for CD8⁺ cytotoxic T-lymphocytes. Antigenic peptides can include peptides having an amino acid sequences corresponding to a sequence located between position 155 and 167 of the NY-ESO-1 protein, where the antigenic peptides can include overlapping sequences. For example, NY-ESO-1 antigens can include a peptide having the amino acid sequence SLLMWITQCFL (SEQ ID NO:2), SLLMWITQC (SEQ ID NO:3), and QLSLLMWIT (SEQ ID NO:4).

NY-ESO-1 peptide antigens can be modified in ways that do not interfere with their ability to generate an immune reaction. For example, NY-ESO-1 peptide antigens can contain, for example, one or more D-amino acids in place of a corresponding L-amino acid; or can contain one or more amino acid analogs, for example, an amino acid that has been derivatized or otherwise modified at its reactive side chain. Similarly, one or more peptide bonds in the NY-ESO-1 peptide antigen can be modified, or a reactive group at the amino terminus or the carboxy terminus or both can be modified. In some embodiments, the NY-ESO-1 peptide antigens can include a C165V substitution. Such modified NY-ESO-1 antigens can have improved ability to bind linkers, as well as improved stability to a protease, an oxidizing agent or other reactive material the polypeptide may encounter in a living subject.

In certain embodiments, the vaccine composition can include a CPMV virus particle conjugated to a plurality of human HLA-A2 restricted peptide antigens having the amino acid sequence corresponding to NY-ESO-1₁₅₇₋₁₆₅ with a C165V substitution (SEQ ID NO: 1). In an exemplary embodiment, chemical fusion can be used to produce CPMV-based NY-ESO-1 anti-cancer particles, where a plurality of NY-ESO-1₁₅₇₋₁₆₅ peptides (SLLMWITQV; SEQ ID NO: 1) are linked to the exterior surface of CPMV virus particles.

The NY-ESO-1 antigen(s) can be linked to the icosahedral-shaped plant virus particle by any suitable technique known to those skilled in the art for linking a peptide and a protein. NY-ESO-1 peptide antigens can be coupled to an icosahedral plant virus particle or virus like particle either directly or indirectly (e.g. via a linker group). The location of the NY-ESO-1 peptide on the exterior can be governed by the amino acids of the viral coat protein, for example, CPMV capsid includes about 300 reactive lysine residues available for bioconjugation.

In some embodiments, the NY-ESO-1 antigens are linked or coupled to the plant virus particle using a linker group. NY-ESO-1 antigens can be conjugated to the plant virus particle by any suitable technique, with appropriate consideration of the need for pharmacokinetic stability and reduced overall toxicity to the patient. A linker group can serve to increase the chemical reactivity of a substituent on either the agent or the virus particle, and thus increase the coupling efficiency, and can also improve the immunogenicity of the linked antigen. In some cases, the linker can include a short spacer consisting of 2 to 10 amino acids (e.g., glycine). For example, the linker group can include a short peptide linker, such as an LSPG peptide linker. Coupling can be affected, for example, through amino groups, carboxyl groups, sulfhydryl groups or oxidized carbohydrate residues. Groups suitable as sites for attaching antigens to the virus particle include lysine residues present in the viral coat protein.

The number of antigens linked to the plant virus particle will vary depending on the number of coat proteins in the plant virus particle, and the availability of suitable reactive groups (e.g., amine, carboxyl, thiol) in the coat proteins. In some embodiments, the plant virus particle is linked to from 2 to 1,000 NY-ESO-1 antigens, while in other embodiments the virus particle is linked to from 5 to 100 NY-ESO-1 antigens, or from 20 to 80 NY-ESO-1 antigens. In certain embodiments, a CPMV anti-cancer particle can include about 30 to about 60 NY-ESO-1 antigen peptides per CPMV.

In some embodiments, a chemical linker group can be used. A linker group can serve to increase the chemical reactivity of a substituent on either the agent or the icosahedral-shaped virus particle or virus-like particle, and thus increase the coupling efficiency. Linkage chemistries include maleimidyl linkers, which can be used to link to thiol groups, isothiocyanate and succinimidyl (e.g., N-hydroxysuccinimidyl (NHS)) linkers, which can link to free amine groups, diazonium which can be used to link to phenol, and amines, which can be used to link with free acids such as carboxylate groups using carbodiimide activation. Cysteine modified antigenic peptides using amine-to-sulfhydryl crosslinkers with aliphatic spacers that differ in chain lengths from 4.4 Angstrom to 9.4 Angstroms or crosslinkers with a PEG spacer varying in lengths form 17.6 Angstroms to 95.2 Angstroms, can also be used. Useful functional groups are present on viral coat proteins based on the particular amino acids present, and additional groups can be designed into recombinant viral coat proteins. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues.

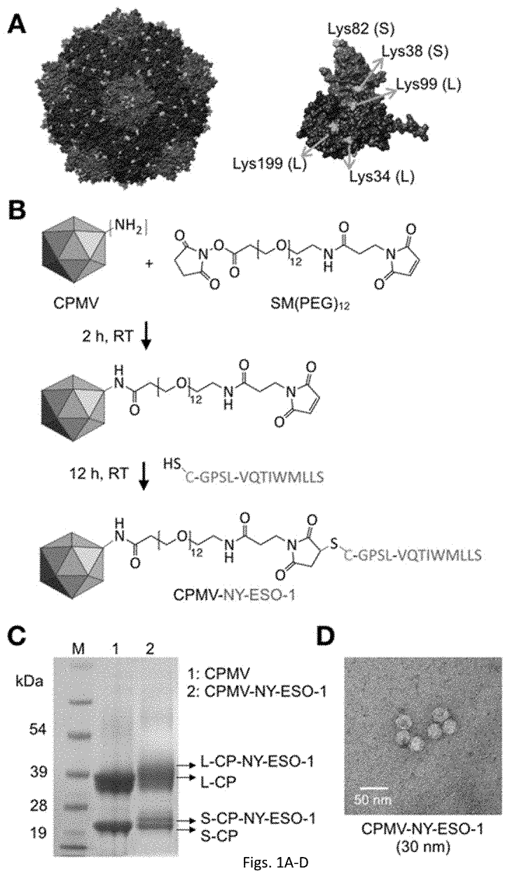

In some embodiments, a NY-ESO-1 peptide having amino acid sequence SLLMWITQV-LSPG-C(SEQ ID NO:5), which includes a flexible LSPG linker and a terminal cysteine, are conjugated to CPMV using a two-step protocol using a bi-functional N-hydroxysuccinimide-PEG12-maleimide (SM-PEG12) linker (see FIG. 1B).

In other embodiments, the NY-ESO-1 antigen is linked to the icosahedral-shaped plant virus particle through expression of a recombinant protein in plants using an N-terminal fusion on the coat protein. Methods for the preparation and isolation of recombinant fusion proteins are well-known to those skilled in the art. For example, in one embodiment, the recombinant polypeptide includes a NY-ESO-1 antigen having an amino acid sequence corresponding to amino acid residues about 157 to 165 of the NY-ESO-1 protein with a C165V substitution (i.e., SEQ ID NO:1). A recombinant polypeptide of the invention can be expressed from a recombinant polynucleotide or can be chemically synthesized. Preparation of recombinant protein antigens are described in U.S. Pat. No. 7,446,185.

In another aspect, the present invention provides a method of treating or decreasing the risk of developing an NY-ESO-1⁺ cancer in a subject in need thereof by administering to the subject in need thereof an effective amount of a vaccine composition comprising an icosahedral plant virus or virus-like particle (VLPs) particle conjugated to a plurality of NY-ESO-1 peptides.

Other embodiments described herein relate to methods of stimulating an immune response in a subject against cells that express NY-ESO-1 antigen. Cells that express NY-ESO-1⁺ can include tumor cancer cells or any other cells that express NY-ESO-1⁺, particularly cells involved in a pathologic condition. NY-ESO-1 is typically expressed during embryonic development until birth in human fetal testis and in spermatogonia and in primary spermatocytes of adult testis. The disclosed methods are particularly useful for stimulating an immune response against cells that are involved in a pathologic condition characterized by aberrant NY-ESO-1 antigen expression, or aberrant re-expression, as compared to corresponding cells that are not involved in the pathologic condition. For example, the cells can be cancer cells that re-express NY-ESO-1 protein as compared to the non-expression by normal cell counterparts to the cancer cells.

Stimulating an immune response in a subject using the vaccine compositions described herein can be used to either treat or prevent cancer, such as NY-ESO-1 expressing cancer. In one embodiment, a method of stimulating an immune response in a subject against cancer cells that express NY-ESO-1 is performed by administering the vaccine composition of under conditions that result in the stimulation of a CD8+ CTL immune response by the vaccine composition against the NY-ESO-1-expressing cells.

When used to treat cancer, the vaccine composition is administered to a subject who has been diagnosed with cancer, in order to stimulate or increase an immune response against the cancer cells. The vaccine composition can be used as the sole method of treatment, or it can be combined with other methods of treating the cancer. Alternately, the vaccine composition can be administered to a subject who has not been diagnosed with cancer as a means of preventing or decreasing the risk or likelihood of cancer development. In some embodiments, the subject being treated and/or immunized using vaccine compositions described herein has been characterized as being a subject having a high or increased risk of developing cancer, such as an NY-ESO-1-expressing cancer. Subjects can be characterized as being at high or increased risk of developing an NY-ESO-1-expressing cancer as a result of, for example, family history, genetic testing, or high exposure to cancer-causing environmental conditions.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

The cancers treated by a method described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, fallopian tube cancer, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In certain embodiments, cancers treated in accordance with a method described herein include NY-ESO-1+ cancers. NY-ESO-1+ cancers can include triple negative breast cancer, melanoma, myelomas and ovarian cancer. In some embodiments, the breast cancer is triple negative breast cancer. In an exemplary embodiment, the cancer is malignant melanoma.

In some embodiments, the subject being administered a therapeutically effective amount of the vaccine composition is a subject who has been identified as having cancer. As is known to those skilled in the art, there are a variety of methods of identifying (i.e., diagnosing) a subject who has cancer. For example, diagnosis of cancer can include one or more of a physical exam, laboratory tests, imaging analysis, and biopsy. After cancer is diagnosed, a variety of tests may be carried out to look for specific features characteristic of different types and or the extent of cancer in the subject. These tests include, but are not limited to, bone scans, X-rays, immunophenotyping, flow cytometry, and fluorescence in situ hybridization testing. For example, typical methods of diagnosing triple-negative breast cancer can include, but are not limited to, a physical exam, digital mammogram, breast MRI, breast ultrasound, stereotactic core and/or open tumor biopsy, as well as lab tests to determine if the tumor tissue expresses estrogen, progesterone, and HER-2/neu or not.

In some embodiments, a method of treating cancer described herein can include administering an additional therapeutic or cancer therapy to the subject. A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy.

In some embodiments, the method can include the step of administering a therapeutically effective amount of an additional anticancer therapeutic agent to the subject. Additional anticancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA. In some embodiments, cytotoxic compounds are included in an anticancer agent described herein. Cytotoxic compounds include small-molecule drugs such as doxorubicin, methotrexate, vincristine, and pyrimidine and purine analogs, referred to herein as antitumor agents.

The additional anticancer therapeutic agent can include an anticancer or an antiproliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be administered in combination with a vaccine described herein include Taxol, Adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; temozolomide, teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In certain embodiments, additional therapeutic agents administered to a subject for the treatment of triple negative breast cancer as described herein can include one or more of an anthracycline, such as adriamycin, an alkylating agent such as Cytoxan (cyclophosphamide), an antimetabolite such as Fluorouracil (5FU), and a taxane, such as Taxol or Taxotere. In other embodiments, additional therapeutic agents administered to a subject for the treatment of melanoma as described herein can include one or more of Aldesleukin, Binimetinib, Braftovi (Encorafenib), Cobimetinib, Cotellic (Cobimetinib), Dabrafenib Mesylate, Dacarbazine, Encorafenib, Imlygic (Talimogene Laherparepvec), Intron A (Recombinant Interferon Alfa-2b), Keytruda (Pembrolizumab), Mekinist (Trametinib), Mektovi (Binimetinib), Nivolumab, Opdivo (Nivolumab), Peginterferon Alfa-2b, PEG-Intron (Peginterferon Alfa-2b), Recombinant Interferon Alfa-2b, Sylatron (Peginterferon Alfa-2b), Tafinlar (Dabrafenib Mesylate), Talimogene Laherparepvec, Trametinib, Vemurafenib, Yervoy (Ipilimumab), and Zelboraf (Vemurafenib).

In some embodiments, the anti-cancer therapy administered to the subject in addition to the vaccine composition can include a cancer ablation therapy. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins. Another method of ablating cancer such as breast cancer that has been treated with an anti-cancer particle composition of the present invention is to conducting surgery to remove the cancer tissue (e.g., breast cancer tissue) from the subject. Types of surgery for breast cancer vary depending on the nature of the breast cancer, and include lumpectomy, partial or segmental mastectomy or quadrantectomy, simple or total mastectomy, radical mastectomy, and modified radical mastectomy. Appropriate surgeries for treating other types of NY-ESO-1⁺ cancer are known to those skilled in the art.

In some embodiments, ablating the cancer includes administering a therapeutically effective amount of radiotherapy (RT) to the subject. In some embodiments, RT is administered prior to administration of the icosahedral-shaped plant virus nanoparticle. In some embodiments, administering to the cancer, (e.g., at a tumor site) a therapeutically effective amount of a icosahedral-shaped plant virus or virus-like particle conjugated to NY-ESO-1 peptide antigen to the subject in combination with administering radiotherapy to the subject can result in an increase in tumor infiltrating lymphocytes (TILs), such as tumor infiltrating neutrophils (TINs) at the tumor site of the subject.

Radiotherapy uses high-energy rays to treat disease, usually x-rays and similar rays (such as electrons). Radiotherapy administered to a subject can include both external and internal. External radiotherapy (or external beam radiation) aims high-energy x-rays at the tumor site including in some cases the peri-tumor margin. External radiotherapy typically includes the use of a linear accelerator (e.g., a Varian 2100C linear accelerator). External radiation therapy can include three-dimensional conformal radiation therapy (3D-CRT), image guided radiation therapy (IGRT), intensity modulated radiation therapy (IMRT), helical-tomotherapy, photon beam radiation therapy, proton beam radiation therapy, stereotactic radiosurgery and/or sterotactic body radiation therapy (SBRT).

Internal radiotherapy (brachytherapy) involves having radioactive material placed inside the body and allows a higher dose of radiation in a smaller area than might be possible with external radiation treatment. It uses a radiation source that is usually sealed in an implant. Exemplary implants include pellets, seeds, ribbons, wires, needles, capsules, balloons, or tubes. Implants are placed in your body, very close to or inside the tumor. Internal radiotherapy can include intracavitary or interstitial radiation. During intracavitary radiation, the radioactive source is placed in a body cavity (space), such as the uterus. With interstitial radiation, the implants are placed in or near the tumor, but not in a body cavity.

In some embodiments, an immune checkpoint inhibitor can be further administered to eradicate suppressive regulatory T cells prior to RT. Exemplary checkpoint inhibitors can include CTLA4 and PD-1/PDL-1 inhibitors. The cytotoxic T-lymphocyte-associated antigen 4 (CTLA-4) and programmed death 1 (PD-1) immune checkpoints are negative regulators of T-cell immune function and inhibition of these targets, results in increased activation of the immune system. Therefore, in some embodiments, a checkpoint inhibitor administered to a subject can include a CTLA-4 and/or PD-1 inhibitor. For example, Ipilimumab, an inhibitor of CTLA-4, is approved for the treatment of advanced or unresectable melanoma. Nivolumab and pembrolizumab, both PD-1 inhibitors, are approved to treat patients with advanced or metastatic melanoma and patients with metastatic, refractory non-small cell lung cancer. In addition, the combination of ipilimumab and nivolumab has been approved in patients with BRAF WT metastatic or unresectable melanoma. In some embodiments, an immune checkpoint agonistic agent, such as an OX40 agonistic agent, can be further administered can be administered promote immune activation of cytotoxic T-cells.

It has been shown that moderate magnetic nanoparticle hyperthermia (mNPH) treatment administered to a tumor can generate immune-based systemic resistance to tumor rechallenge. Therefore, in some embodiments, a therapeutically effective amount of a moderate magnetic nanoparticle hyperthermia (mNPH) treatment can be administered to the subject in combination with an anti-cancer plant virus particle or virus-like particle and/or radiotherapy, wherein the mNPH is activated with an alternating magnetic field (AMF) to produce moderate heat. Without being bound by theory, it is believed that plant virus-like particle immune adjuvants, such as a plant virus nanoparticles described herein and/or a mNPH, will combine with RT-induced generation of immunogenic cell death (ICD) to expand the tumor specific effector T cell population causing longer local and distant tumor remission.

A mNPH treatment can include the use of a magnetic iron oxide nanoparticle (IONP). Once administered to the subject intratumorally, the mNPH can, in some embodiments, be activated with an alternating magnetic field (AMF) to produce moderate heat (e.g., 43°/60° min) at the tumor site. In some embodiments, the RT is hypofractionated RT (HFRT) that delivers larger but fewer doses/fractions than typical RT therapies.

In order to evaluate the efficacy of the NY-ESO-1 vaccine composition described herein, challenge studies can be conducted. Such studies involve the inoculation of groups of test animals (such as mice) with a vaccine composition described herein by standard techniques. Control groups comprising non-inoculated animals and/or animals inoculated with a commercially available vaccine, or other positive control, are set up in parallel. After an appropriate period of time post-vaccination, the animals are challenged with a cancer cells. Blood samples collected from the animals pre- and post-inoculation, as well as post-challenge are then analyzed for an antibody response and/or T cell response to the NY-ESO-1 antigen. Suitable tests for the T and B cell responses include, but are not limited to, Western blot analysis and Enzyme-Linked Immunosorbent Assay (ELISA) assay. Cellular immune response can also be assessed by techniques known in the art, including monitoring T cell expansion and IFN-γ secretion release, for example, by ELISPOT to monitor induction of cytokines.

The animals can also be monitored for development of other conditions associated with infection with cancer including, for example, growing tumor size, and the like for certain cancer cell lines, survival is also a suitable marker.

When used in vivo, the vaccine composition described herein can be administered as a pharmaceutical composition, comprising a mixture, and a pharmaceutically acceptable carrier. The vaccine composition may be present in a pharmaceutical composition in an amount from 0.001 to 99.9 wt %, more preferably from about 0.01 to 99 wt %, and even more preferably from 0.1 to 95 wt %.

The vaccine composition may be administered by any method designed to provide the desired effect. Administration may occur enterally or parenterally; for example orally, topically, rectally, intracisternally, intravaginally, intraperitoneally or locally. Parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device. In some embodiment, the anti-cancer particles may be administered topically. Anti-cancer particles can be topically administered passively for example, by direct application of an ointment or a skin patch, or administered actively, for example, using a nasal spray or inhalant, in which case one component of the composition is an appropriate propellant or through the use of facilitated absorption through the skin using, for example, transdermal iontophoresis.

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The pharmaceutical compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers and the like.

Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

Suitable doses can vary widely depending on the therapeutic being used. A typical pharmaceutical composition for intravenous administration would be about 0.1 mg to about 10 g per subject per day. However, in other embodiments, doses from about 1 mg to about 1 g, or from about 10 mg to about 1 g can be used. Single or multiple administrations of the compositions may be administered depending on the dosage and frequency as required and tolerated by the subject. In any event, the administration regime should provide a sufficient quantity of the composition of this invention to effectively treat the subject.

Useful dosages of the additional anticancer agents, such as antimitotic agents, and anti-cancer plant virus particles can be determined by comparing their in vitro activity and the in vivo activity in animal models. Methods for extrapolation of effective dosages in mice, and other animals, to humans are known in the art; for example, see U.S. Pat. No. 4,938,949. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the additional anticancer agents and/or anti-cancer plant virus particles vary depending upon many different factors, including means of administration, target site, physiological state of the patient, whether the patient is human or an animal, other medications administered, and whether treatment is prophylactic or therapeutic.

The formulations may be conveniently presented in unit dosage form and may be prepared by any of the methods well known in the art of pharmacy. Preferably, such methods include the step of bringing the plant virus particles into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the vaccine composition in an amount effective to produce the desired effect.

One skilled in the art can readily determine an effective amount of the vaccine composition to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, suitable doses of the anti-cancer virus particles to be administered can be estimated from the volume of cancer cells to be killed or volume of tumor to which the virus particles are being administered.

Useful dosages of the vaccine composition can be determined by comparing their in vitro activity and the in vivo activity in animal models. Methods for extrapolation of effective dosages in mice, and other animals, to humans are known in the art. An amount adequate to accomplish therapeutic or prophylactic treatment is defined as a therapeutically- or prophylactically-effective dose. In both prophylactic and therapeutic regimes, the vaccine composition can be administered in several dosages until an effect has been achieved. Effective doses of the vaccine composition vary depending upon many different factors, including means of administration, target site, physiological state of the patient, whether the patient is human or an animal, characteristics of the subject, such as general health, age, sex, body weight and tolerance to drugs as well as the degree, severity and type of cancer, other medications administered, and whether treatment is prophylactic or therapeutic. The skilled artisan will be able to determine appropriate dosages depending on these and other factors using standard clinical techniques. In some embodiments, the therapeutically effective amount of vaccine composition described herein is the amount effective to enhance uptake and activation of antigen presenting cells and promote a potent CD8+ T cell response in the subject.

The methods described herein contemplate single as well as multiple administrations, given either simultaneously or over an extended period of time. A pharmaceutically acceptable composition containing the vaccine composition can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the pharmaceutically acceptable composition containing the anti-cancer plant virus particles and/or an additional cancer therapeutic is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased.

For example, the administration of the vaccine composition and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

In an exemplary embodiment, the vaccine composition is administered to the subject in need thereof via subcutaneous injection twice in two weeks to immunize the subject.

In some embodiments, the frequency of administration of the vaccine composition can pose challenging for clinical implementation. Therefore, in some embodiments, the vaccine composition administered to a subject can be formulated in a slow release formulation in order to sustain immune stimulation by maintaining a therapeutic concentration of the vaccine compositions while alleviating the need for frequent administrations. In some embodiments, a slow release formulation can include a polymer-based hydrogel or a dendrimer.

In some embodiments, a slow-release formulation can include an anti-cancer plant virus or plant virus like particle dendrimer hybrid aggregate. The dendrimer can include a positively-charged polyamidoamine (PAMAM) dendrimer, such as a medium-sized generation 3 (G3) or generation 4 (G4) PAMAM dendrimer. Depending on the specific application, the plant virus-like particle-dendrimer hybrid aggregates can vary in size and release rate of the plant virus-like particle from the dendrimer when administered to a subject. In some embodiments, the anti-cancer plant virus particle-dendrimer hybrid aggregates are formulated so that at low salt the assembly of the aggregates is triggered and while under physiologic salt concentrations disassembly and anti-cancer plant virus particle release is induced.

Examples have been included to more clearly describe particular embodiments of the invention. However, there are a wide variety of other embodiments within the scope of the present invention, which should not be limited to the particular examples provided herein.

### Example

In this Example, we exploited a plant virus epitope display platform technology-cowpea mosaic virus (CPMV)—to develop an NY-ESO-1 vaccine. Here, CPMV serves the dual purpose of a delivery system and an adjuvant. The 30 nm icosahedral ssRNA viral nanoparticle of CPMV has been previously established as a highly potent antigenic carrier and immune stimulant. The potency of CPMV as an immune stimulant is derived from its highly organized three-dimensional (3D) protein architecture with its encapsidated nucleic acid and an intrinsic immune cell tropism. We have established that CPMV can facilitate efficient delivery of tumor antigens to antigen-presenting cells (APCs) and provide the additional immune stimulus for effective processing and presentation of these antigens. Here, the motivation was to develop a CPMV-based vaccine to stimulate an antigen specific cellular immune response. To generate an effective cytotoxic T cell response against cancer antigen using a cancer vaccine, exogenous peptide epitopes must be delivered to the cytosol of antigen-presenting cells (APCs) for cross-presentation. Here, CPMV was chemically modified to display multiple copies of the HLA-A2-restricted NY-ESO-1157-165 peptide. The potency CPMV-NY-ESO-1 vaccine to stimulate an antigen-specific CTL response was then tested in transgenic human HLA-A2 expressing mice.

### Materials and Methods

### Production of Cowpea Mosaic Virus (CPMV)

CPMV was propagated in V. unguiculata plants and purified from infected leaves using previously described methods. Post-purification CPMV concentrations were determined by ultraviolet/visible (UV/vis) spectroscopy (CPMV specific extinction coefficient ε_{260 nm}=8.1 mg⁻¹ mL cm⁻¹). Particle integrity was verified by the elution profile determined by size exclusion chromatography using a Superose6 column on the ÄKTA Explorer chromatography system (GE Healthcare, Pittsburgh, Pa.) and the 260:280 ratio (for intact CPMV the 260:280 ratio is 1.8).

### Synthesis of CPMV-NY-ESO-1 Vaccine

NY-ESO-1 peptide (NY-ESO-1₁₅₇₋₁₆₅) with a terminal cysteine and a flexible LSPG linker-SLLMWITQV-LSPG-C, or its fluorescent version-tetramethylrhodamine (TMR)-conjugated peptide NY-ESO-1_{TMR} (Genscript, Piscataway, N.J.), was conjugated to CPMV using a two-step protocol through a bifunctional N-hydroxysuccinimide-PEG12-maleimide (SMPEG12) linker (Thermo Fisher Scientific, Waltham, Mass.). CPMV in 0.1 M sodium phosphate (KP) buffer pH 7.4 with constant mixing was first reacted with 2000 molar excess of the SM-PEG12 linker at room temperature for 2 h at 1 mg/mL protein concentration. Next, 3000 molar excess of the peptide was reacted overnight with SMPEG12-modified CPMV. The CPMV-NY-ESO-1 formulation was purified by spin filtration (10 000 rpm/5 min; 100 kDa molecular weight cutoff filters, Amicon Ultra, Millipore Sigma, Burlington, Mass.).

Conjugation of NY-ESO-1 peptides on CPMV was quantified using SDS-PAGE gels. Ten micrograms of CPMV and CPMV-NY-ESO-1 was mixed with SDS running buffer (Thermo Fisher Scientific), heated at 100° C. for 5 min, and then loaded on precast Nu-PAGE 4-12% Bis-Tris protein gels (Thermo Fisher Scientific). Electrophoresis was performed for 40 min at 200 V. Gels were stained using GelCode Blue Safe protein stain (Thermo Fisher Scientific) to visualize the protein bands corresponding to molecular weight ladders; the degree of peptide modification was quantified using lane density analysis (Fiji software). Particle integrity was verified by transmission electron microscopy (TEM). CPMV-NY-ESO-1 particles (0.5 mg/mL) were loaded on 400-mesh copper grids bearing the Formvar support film, stained with 2% (w/v) uranyl acetate, and visualized using the FEI Tecnai Spirit G2 BioTWIN microscope (FEI, Hillsboro, Oreg.). CPMV-NY-ESO-1-TMR formulation was characterized using denaturing and native gel electrophoresis. Native gel electrophoresis (100 V for 40 min) of CPMV and CPMV-NY-ESO-1− TMR particles (10 μg in 6× loading dye) was performed on agarose gel (1.2% w/v) containing 1 μL of GelRed Nucleic Acid Stain for RNA visualization (GoldBio, St Louis, Mo.) in Tris borate EDTA (TBE) buffer. The native gel was visualized under UV light for nucleic acid, using 534 nm light source for TMR dye and after staining with Coomassie Brilliant Blue (0.25% w/v) (Sigma, St Louis, Mo.) to visualize the capsid protein; denaturing gel was visualized under 534 nm for fluorescence and white light for stained proteins. CPMV-NYESO-1-TMR was also characterized using size exclusion chromatography using a Superose6 column on the ÄKTA Explorer chromatography system (GE Healthcare, Marlborough, Mass.).

A CPMV-OVA vaccine was similarly synthesized by conjugating the H2-Kb-restricted OVA peptide with the GPSL linker and a terminal cysteine C-LSPG-SIINFEKL (Genscript) to CPMV via the SM-PEG12 linker.

### Mice

All animal experiments were carried out in accordance with Case Western Reserve University's Institutional Animal Care and Use Committee (IACUC). Transgenic C57BL/−Mcph1 Tg (HLA-A2.1)-1Enge/J mice, expressing the human HLA-A2 gene (HLA-A2 mice), were obtained from the Jackson Laboratory (Bar Harbor, Me.). Six-eight week old female HLA-A2 mice were immunized subcutaneously twice at 14 days intervals with 50 μg of CPMV-NY-ESO-1 vaccine in 100 μL of phosphate-buffered saline (PBS) or equivalent dose of the NY-ESO-1 peptide mixed with Complete Freud's Adjuvant (CFA) for first injection/Incomplete Freund's Adjuvant (IFA) for booster injection (Invivogen, San Diego, Calif.) in 100 μL of PBS. To evaluate the CPMV-OVA vaccine, 8-week-old female C57BL6 mice (Jackson Laboratory) were immunized with 50 μg of CPMV-OVA or 1 μg of OVA peptide, as described for the NY-ESO-1 antigen. Two weeks after the last immunization, mice were sacrificed and spleens were harvested to isolate CD8+ T cells.

### Cell Lines

A375, an HLA-A2+NY-ESO-1+ human malignant melanoma cell line, was purchased from ATCC (Manassas, Va.) and cultured in Dulbecco's modified Eagle's medium (DMEM) supplemented with 10% (v/v) fetal bovine serum (FBS) (Atlanta Biologicals, Minneapolis, Minn.) and 1% (v/v) penicillin/streptomycin (Pen/Strep) (Thermo Fisher Scientific). Murine macrophage cell lines RAW 264.7, mouse melanoma cells B16F10 (ATCC), and B16F10-OVA (gift from Dr. Steve N. Fiering, Dartmouth College, NH) were maintained on the DMEM medium described above.

### Macrophage Uptake

Macrophage uptake of CPMV-ESO-1TMR particles and soluble ESO-1TMR peptides was compared using confocal microscopy. Thirty thousand RAW 264.7 macrophage cells in 0.5 mL media were seeded overnight in 24-well plates on circular glass coverslips. Cells were incubated with 5 μg of CPMV-NY-ESO-1TMR particles or 1 μg of NY-ESO-1TMR peptides for 2 h at 37° C. Cells were washed, fixed in 5% (v/v) paraformaldehyde/0.3% (v/v) glutaraldehyde in Dulbecco's phosphate-buffered saline (DPBS) for 10 min, stained for the cell membrane with Wheat Germ Agglutinin-AlexaFluor 488 (Thermo Fisher Scientific), and diluted 1:1000 in 5% (v/v) goat serum (Thermo Fisher Scientific) in DPBS. Nuclei were stained with DAPI in the mounting medium (Vector Laboratories, Burlingame, Calif.). The stained cells were imaged on a Leica TCS SPE confocal microscope with a 63× oil immersion objective and images were analyzed with Fiji software.

### Bone Marrow-Derived Dendritic Cells (BMDCs)

BMDCs were isolated from a single-cell suspension of whole bone marrow cells harvested from the femurs and tibias of female HLA-A2 or C57BL6 mice. The cells were washed with PBS, and red blood cells were lysed using RBC lysis buffer (Thermo Fisher Scientific) at 37° C. for 5 min. The cells were then centrifuged, washed, and resuspended at 3×106 cells/mL in the T cell medium: Roswell Park Memorial Institute (RPMI) (Thermo Fisher Scientific) supplemented with 10% (v/v) GemCell FBS (Gemini Bio-Products, West Sacramento, Calif.), 1% (w/v) Pen/Strep, 1 mM sodium pyruvate (Thermo Fisher Scientific), and 50 mM β-mercaptoethanol (Millipore Sigma) supplemented with 10 ng/mL mouse IL-4 and 15 ng/mL mouse GM-CSF (both Peprotech, Rocky Hill, N.J.). The media was removed and replaced with fresh T cell media supplemented with IL-4 and GM-CSF on day 3 and then again on day 5. Cells were harvested on day 7 and used for BMDC activation and antigen-presentation studies.

### BMDC Activation and Cytokine Secretion

BMDCs harvested on day 7 were plated at 1×10⁶ cells/100 μL medium and incubated with 10 μg of CPMV-NY-ESO-1 particles and 2 μg of the NY-ESO-1 peptide (10× equivalent NY-ESO-1 peptide compared to CPMV-NYESO-1) at 37° C. for 24 h in cytokine-free T cell media. Bacterial LPS (100 ng/mL, eBioscience, Thermo Fisher Scientific) was used as a positive control. Following incubation, cell supernatants were collected and analyzed for cytokines TNF-α, IL-6, IL-12, and IL-1β using ELISA kits (BioLegend, San Diego, Calif.) as per instructions from the manufacturer.

### CD8+ T Cell Proliferation and Stimulation

CD8+ T cells were isolated from single-cell suspension obtained from the spleens of mice immunized with CPMV-NY-ESO-1 and CFA+NY-ESO-1 peptides. Spleens were homogenized and passed through a 40 m cell strainer in ice-cold PBS and centrifuged at 500 g for 5 min. RBCs were depleted with RBC lysing buffer (eBioscience, Thermo Fisher Scientific), and CD8+ T cells were isolated using the RoboSep CD8+ T cell negative isolation kit (STEMCELL Technologies, Cambridge, Mass.) according to the manufacturer's instructions.

CD8+ T cells were co-cultured with antigen-pulsed BMDCs to measure proliferation and IFN-γ secretion. Specifically, BMDCs isolated from naive HLA-A2 mice were pulsed with increasing concentrations of NY-ESO-1 peptide (10, 20, 30 μg/mL) for 4 h at 37° C. Cells were washed twice with PBS to remove excessive peptides. CD8+ T cells isolated from immunized mice (CPMV-NY-ESO-1 and CFA+NY-ESO-1 groups) were co-cultured with antigen-pulsed and nonpulsed BMDCs (stimulating cells) at a 1:10 (T cells/BMDCs) ratio in a 96-well plate in triplicate at 37° C. for 48 h in an atmosphere of 5% CO. CD8+ T cells incubated with CD8+ T cells incubated with the NY-ESO-1 peptide alone were used as controls. T cell proliferation was measured using MTT assays, performed as per the manufacturer's recommendation. A Tecan microplate reader was used for readout. The % cell proliferation was calculated as experimental proliferation/control proliferation×100, where experimental proliferation is the proliferation of co-cultured cells minus proliferation of BMDCs only minus proliferation of T cells only and control proliferation is the proliferation of T cells only. For measurements of secreted IFN-γ, culture supernatants were collected and assayed with the Mouse IFN-γ ELISA kits (BioLegend) as per instructions from the manufacturer.

To determine the epitope specificity, CD8+ T cells from CPMVNY-ESO-1-immunized mice were co-cultured with BMDCs pulsed with NY-ESO-1 peptide or irrelevant HER2 peptide P4 (PESFDGDPASNTAPLQPEQLQ). Secreted IFN-γ levels were compared, as described above.

CD8+ T cells from CPMV-OVA-immunized mice were similarly harvested and secreted IFN-γ levels were measured by co-culturing CD8+ T cells with OVA/P4-pulsed BMDCs from naive C57BL6 mice.

### CD8+ T Cell-Mediated Cancer Cell Cytotoxicity

CD8+ T cells from immunized mice were plated in 96-well plates in RPMI at 5×106 cells/mL and incubated overnight at 37° C. On day 1, 10 μg/mL NY-ESO-1 was added to the cells and incubated at 37° C. Cells were collected on day 3 to perform the cytotoxicity assay. To examine antigen-specific cytotoxicity, NY-ESO-1+ target cancer cells A375 and NY-ESO-1-control cell line B16F10-OVA were co-cultured with CD8+ T cells at effector-to-target ratios of 100:1, 75:1, 50:1, 25:1, 0:1 and cytotoxicity evaluated using the MTT assay. Percentage cytotoxicity was determined as experimental cytotoxicity/control toxicity×100, where experimental cytotoxicity is cytotoxicity for coculture (cytotoxicity of CD8+ T cells only+Cytotoxicity of cancer cells only) and control cytotoxicity is the cytotoxicity of cancer cells only under identical culture conditions. Cytotoxicity of CD8+ T cells from CPMV-OVA/CFA+OVA-immunized mice was similarly evaluated against OVA+B16F10-OVA and OVA-B16F10 cells.

### Results and Discussion

CPMV is a 30 nm icosahedral viral particle with a pseudo T=3 symmetry and consists of 60 copies of a 24 kDa small coat protein (S-CP) and 42 kDa large coat protein (L-CP) (FIG. 1A). A CPMV capsid offers 300 reactive lysines available for bioconjugation using the N-hydroxysuccinimide (NHS) chemistry. CPMV was produced in Vigna unguiculata plants with yields of 50-100 mg of virus/100 g of infected leaves. NYESO-1157-165 (SLLMWITQC) is a validated immunodominant MHC-I epitope that has been extensively studied for the development of subunit vaccines. NY-ESO-1 peptide with a C165V substitution (as previously described), flexible LSPG linker, and a terminal cysteine (SLLMWITQV-LSPG-C) was conjugated to CPMV using a two-step protocol through a bifunctional NHS-maleimide (SM-PEG12) linker (FIG. 1B). Conjugation of NY-ESO-1 peptide on CPMV was confirmed by denaturing sodium dodecyl sulfate polyacrylamide gel electrophoresis (SDS-PAGE) of purified CPMV-NY-ESO-1 particles, which revealed the presence of higher molecular weight protein bands for both the S and L proteins. This is as expected because the Lys side chains are available on both CPMV capsid proteins. The higher molecular weight band above the 24 kDa small coat protein (S-CP) band of CPMV was better resolved, which allowed us to quantify the bands using the ImageJ band analysis tool (FIG. 1C). Density analysis indicated ˜45% of S-CP modified with the NY-ESO-1 peptide (or 27 NY-ESO-1 peptides per S-CP); the measurement on the L-CP was not conclusive because the bands of free and modified L-CP were not sufficiently separated. Therefore, we estimate that on an average 30-60 peptides were displayed per CPMV. While using over a thousand fold molar excess of reacting ligands maximizes uniform conjugation densities, VNP heterogeneity cannot be totally ruled out. Alternatively, genetic engineering methods can be used for the expression of epitopes on CPMV. However, genetic engineering and chemical bioconjugation each have their advantages: for initial development, chemical bioconjugation is the preferred method because of its ease and speed. While some degree of heterogeneity cannot be ruled out, there is no indication that this is a barrier for clinical development as several VLPs formulated using these principles have been in clinical trials. Genetic engineering may allow for a greater degree of homogeneity; however, the development timeline is longer and more cumbersome. Finally, homogeneity is not guaranteed, as previous studies indicated that inserted peptides could be cleaved resulting in loss of the peptides.

Fluorescent NY-ESO-1TMR peptide was similarly conjugated to CPMV and characterized using native and denaturing gel electrophoresis and size exclusion chromatography. The association of fluorescent peptide with the capsid protein was confirmed by the native gel and SDS-PAGE, where CPMV capsid/coat protein bands are fluorescently tagged. The co-elution of 550 nm fluorescent peak with the 260/280 peaks representing the CPMV also confirmed the association of NY-ESO-1-TMR peptide with CPMV capsid. TEM images confirmed the structural integrity of the purified CPMV-NY-ESO-1 particles (FIG. 1D).

As an epitope display platform, the highly ordered 3D architecture of plant virus CPMV offers several unique features. The proteinaceous scaffold allows for a multivalent and repetitive display of the antigenic epitope, which activates pathogen-associated molecular pattern (PAMP) recognition pathways leading to the induction of stronger and longer lasting antigen-specific immune responses. The viral nucleocapsid itself engages several pattern recognition receptors (PRRs) on immune cells, thereby enhancing the immunological visibility of the vaccine and providing additional immune stimulus. Based on these features, we anticipated that CPMV would be a suitable platform technology for the NY-ESO-1 display to launch a potent CD8+ CTL response.

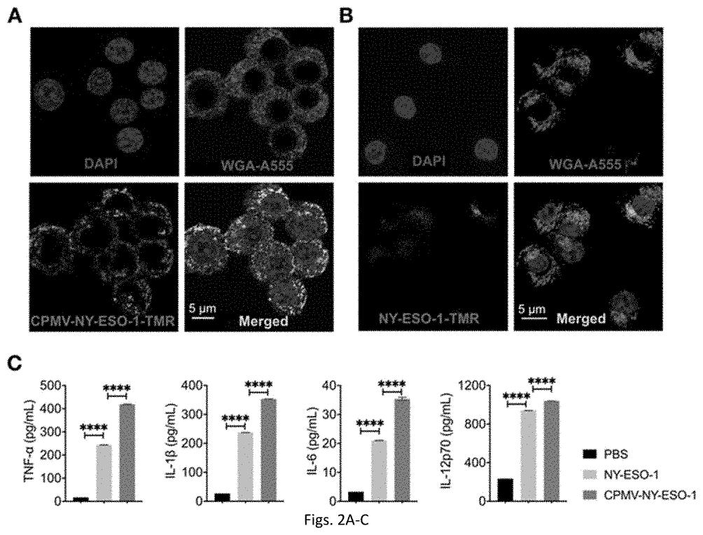

We first evaluated the delivery of the NY-ESO-1 peptide antigen to APCs with and without the CPMV carrier and compared their activation. RAW 264.7 macrophages were incubated with CPMV-NY-ESO-1TMR or equivalent concentration of soluble NY-ESO-1TMR peptide, and cellular uptake was compared using confocal microscopy. As evident from imaging data, a significantly higher uptake of CPMV conjugated NY-ESO-1TMR was observed over soluble nonconjugated NY-ESO-1TMR peptide (FIG. 2A, B). The data corroborated previously observed enhanced delivery of epitopes to APCs by CPMV nanocarriers. Earlier studies have revealed a natural tropism of CPMV for APCs. We have also demonstrated that subcutaneously injected CPMV particles form local depots at the site of administration for sustained trafficking to the draining lymph nodes, thereby extending antigen sampling by peripheral APCs and improving the delivery of antigen to the draining lymph nodes. Together with efficient trafficking, the cellular uptake of the CPMV carrier improves the delivery of tumor antigens to APCs for effective processing and presentation.

To investigate the potency of the CPMV-NY-ESO-1 vaccine to activate APCs, we measured the release of cytokines TNF-α, IL-1β, IL-6, and IL-12p70 by mouse BMDCs upon stimulation with CPMV-NY-ESO-1 versus soluble NY-ESO-1 peptide antigen. Following 24 h incubation with the CPMV-NY-ESO-1 vaccine or equivalent amount of soluble peptide, culture supernatant from mouse BMDCs was analyzed for abovementioned cytokines using ELISAs. NY-ESO-1 peptide and the CPMV-NY-ESO-1 vaccine both resulted in increased production of cytokines over nonstimulated BMDCs, highlighting the potency of the NY-ESO-1 antigen. However, CPMV-NY-ESO-1 significantly enhanced the cytokine levels, with nearly 2-fold higher levels of TNF-α, 1.5-fold higher levels of IL-1β, 1.7-fold higher IL-6, and ˜1.2-fold higher levels of IL-12p70 over the soluble NY-ESO-1 peptide (FIG. 2C).

DCs play a central role in initiating an antigen-specific immune response by presenting antigens to T cells and providing the required immune stimulus through cell-to-cell contact and secreted cytokines. TNF-α plays an important role in maturation and migration of DCs to lymph nodes after sampling antigens and thus is critical for subsequent presentation of the antigens to T cells. TNF-α also enhances local inflammatory responses and plays an essential role in the inhibition of tumor growth. IL-1β release by DCs is induced by the activation of the NLRP3 inflammasome, an intracellular multiprotein signaling complex assembled as an inflammatory response to internalization of certain types of particulate antigens in dendritic cells. IL-1β signaling activates innate immune cells and is critical for T cell priming by dendritic cells. Proinflammatory cytokines IL-6 and IL-12 produced by activated DCs provide the necessary signal to induce the development of CTL effector functions. IL-6 plays a key role in promoting T cell trafficking to lymph nodes for activation and to tumor sites for effector functions. IL-12 links innate and adaptive immune responses. IL-12 released by APCs induces activation and proliferation of NK cells and T cells, polarizes T cells to a type 1 helper T (Th1) effector cell phenotype, and induces production of IFN-γ as primary antitumor response.

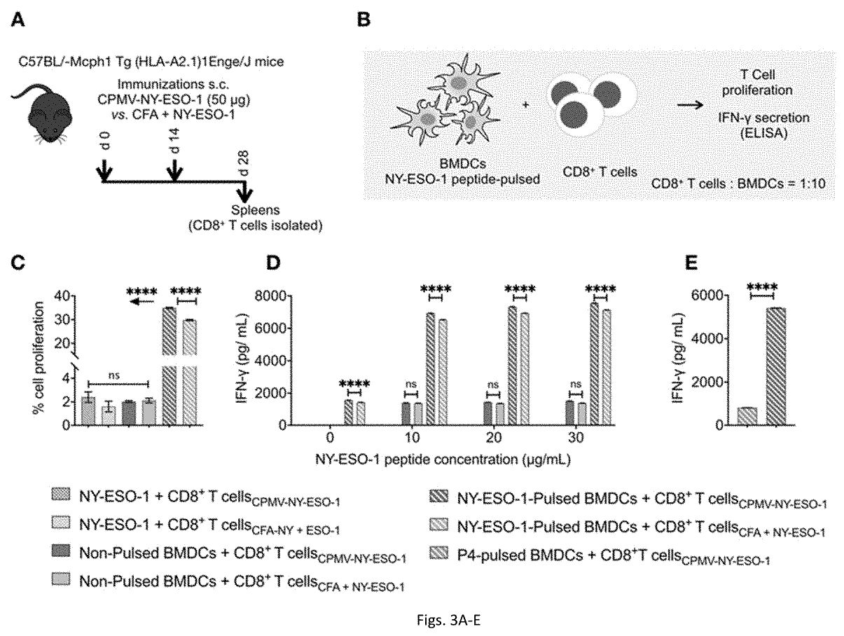

We next investigated the efficacy of the CPMV-NY-ESO-1 vaccine to elicit an antigen-specific T cell response using transgenic mice expressing human HLA-A2, as NY-ESO-1157-165 is an HLA-A2-restricted antigen. Soluble peptides are poorly immunogenic in vivo, therefore the efficacy of the CPMV-NY-ESO-1 vaccine was compared to the equivalent amount of NY-ESO-1 peptide administered with a commercial adjuvant Complete Freund's Adjuvant (CFA). Female HLAA2 mice were immunized subcutaneously with CPMV-NYESO-1 or CFA+NY-ESO-1 at day 0 and with a booster dose of CPMV-NY-ESO-1 or IFA (Incomplete Freund's Adjuvant)+NY-ESO-1 on day 14 (FIG. 3A). Two weeks after the second immunization, spleens were harvested from immunized mice and CD8+ T cells were isolated from the splenocytes. To probe antigen specificity, CD8+ T cell proliferation and activation were evaluated in the presence of NY-ESO-1 peptide-pulsed BMDCs isolated from nai\ddot{v}e HLA-A2 mice (FIG. 3B). CD8+ T cells incubated with nonpulsed BMDCs or with NY-ESO-1 peptide were used as controls. Improved antigen trafficking, APC uptake, and activation facilitated by CPMV translated into an effective cellular immune response. Thus, immunizations with the CPMV-NY-ESO-1 vaccine significantly increased the NY-ESO-1-specific CD8+ T cell population in spleens as evident from the enhanced proliferation and elevated IFN-γ secretion by CD8+ T cells cultured with peptide-pulsed BMDCs as compared to CD8+ T cells incubated with peptide alone or with nonpulsed BMDCs (FIGS. 3C, D). We also observed high-peptide-specific CD8+ T cell proliferation and IFN-γ secretion for the group immunized with NY-ESO-1 peptide+CFA, an adjuvant which is known to induce a strong Th1-dominated inflammatory response. However, the CD8+ T cells from CPMV-NY-ESO-1-immunized mice displayed significantly higher proliferation and IFN-γ secretion compared to those from CFA+NY-ESO-1-immunized mice, suggesting enhanced potency of the CPMV-based vaccine. Furthermore, CD8+ T cell from CPMV-NY-ESO-1 also showed ˜6-fold higher IFN-γ levels when incubated with the NY-ESO-1-pulsed BMDCs as compared to an irrelevant HER2-derived P4 peptide-pulsed BMDCs, suggesting antigen specificity (FIG. 3E).

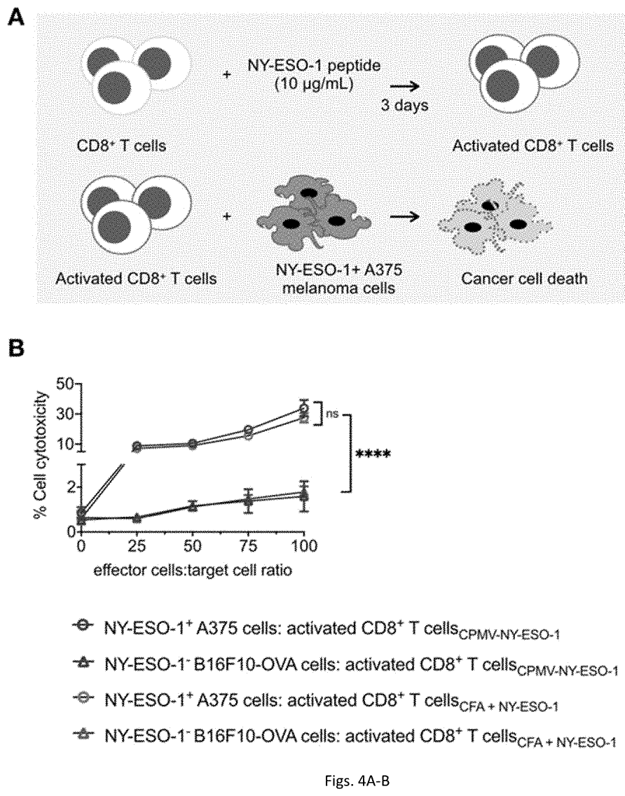

We next evaluated the cytotoxicity of CD8+ T cells toward NY-ESO-1-expressing cancer cells. The elevated levels of peptide-specific IFN-γ translated into significantly higher effector/target cell ratio-dependent cancer cell cytotoxicity. Thus, CD8+ T cells from immunized mice (both the CPMVNY-ESO-1 vaccine and CFA+NY-ESO-1 groups) showed significant cancer cell cytotoxicity when co-cultured with NYESO-1+/HLA-A2-expressing A375 human melanoma cells as compared to NY-ESO-1− mouse B16F10-OVA melanoma cells, which also did not express the HLA-A2 antigen (FIG. 4). Similar studies with a CPMV-OVA vaccine formulation revealed an antigen-specific cellular immune response in C57BL6 mice. Here, CD8+ T cells from immunized mice displayed antigen-specific IFN-γ secretion when co-cultured with OVA-pulsed BMDCs from nai\ddot{v}e C57BL6 mice and resulted in B16F10-OVA cell lysis.

Our results are comparable to those obtained with the protein nanoparticle pyruvate dehydrogenase (E2 nanoparticle)-based NY-ESO-1 vaccine that has been shown to improve DC activation and antigen cross-presentation. However, while the E2 nanoparticle-based NY-ESO-1 vaccine required an additional immune stimulant in the form of the TLR9 agonist CpG oligonucleotide (ODN) 1826 for efficacy, the CPMV viral nanoparticle itself was able to achieve comparable outcomes.

We also observed a comparable antigen-specific cancer cell cytotoxicity with the CFA+NY-ESO-1 vaccine, suggesting similar potency of CPMV and CFA as adjuvants. Similar to other adjuvants, CFA is likely to stimulate the immune system via multiple mechanisms including depot effect, recruiting APCs to the site of injection, enhancing antigen uptake, APC activation, and migration of activated APCs to draining lymph nodes. While CFA has been proven an effective Th1 adjuvant for preclinical research, its use is associated with strong long lasting and possibly painful local inflammation. Therefore, the application of CFA as an adjuvant is restricted by numerous regulatory guidelines. IFA, used for booster immunizations, has been tested in clinical trials but was discontinued as a vaccine adjuvant in humans due to associated severe side effects. Therefore, there is a need for the development of novel adjuvants, and our data support the need for further development of the CPMV platform for cancer immunotherapy applications. Other clinically approved adjuvants such as monophosphoryl lipid A (MPL), MF59, and alum have been previously evaluated for cancer vaccines, often requiring additional immunostimulatory molecules to achieve a Th1 immune response (for example, CpG ODN). CPMV, on the other hand, serves the dual purpose of a carrier and an adjuvant, with established Th1 immune response. Thus, the CPMV-delivering platform obviates the need for additional immunostimulants.

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A vaccine comprising an icosahedral-shaped plant virus or virus-like particle linked to a plurality of NY-ESO-1 antigens.

2. The vaccine composition of claim 1, wherein the plant virus or plant virus-like particle is of the Secoaviridae family.

3. The vaccine composition of claim 1, wherein the plant virus or plant virus-like particle is of the genus Comovirus.

4. The vaccine composition of claim 1, wherein the plant virus or plant virus-like particle is a cowpea mosaic virus (CPMV) or CPMV virus-like particle.

5. The vaccine composition of claim 1, wherein the NY-ESO-1 antigen comprises all or a portion of a peptide having the amino acid sequence located between position 155 and 167 of the NY-ESO-1 protein.

6. The vaccine composition of claim 5, wherein the NY-ESO-1 antigen comprises a peptide having the amino acid sequence selected from SLLMWITQCFL (SEQ ID NO:2), SLLMWITQC (SEQ ID NO:3), and QLSLLMWIT (SEQ ID NO:4).

7. The vaccine composition of claim 5, wherein the NY-ESO-1 antigen comprises a peptide having the amino acid sequence SLLMWITQV (SEQ ID NO:1).

8. The vaccine composition of claim 5, wherein the antigen comprises a cysteine terminated NY-ESO-1 peptide with an intervening flexible linker.

9. The vaccine composition of claim 8, wherein the NY-ESO-1 antigen comprises a peptide having the amino acid sequence SLLMWITQV-LSPG-C (SEQ ID NO:5)

10. The vaccine composition of claim 1, further comprising a pharmaceutically acceptable carrier.

11. The vaccine composition of claim 1, wherein the plurality of NY-ESO-1 antigens are conjugated to the external surface of the plant virus or plant virus-like particle.

12. A method of treating or decreasing the risk of developing a NY-ESO-1-expressing cancer in a subject in need thereof, the method comprising:
administering to the subject a therapeutically effective amount of vaccine composition comprising an icosahedral-shaped plant virus or virus-like particle linked to a plurality of NY-ESO-1 antigens.

13. The method of claim 12, wherein the plant virus or plant virus-like particle is a cowpea mosaic virus (CPMV) or virus-like particle.

14. The method of claim 12, wherein the NY-ESO-1 antigen comprises a peptide having an amino acid sequence selected from SLLMWITQCFL (SEQ ID NO:2), SLLMWITQC (SEQ ID NO:3), and QLSLLMWIT (SEQ ID NO:4).

15. The method of claim 12, wherein the NY-ESO-1 antigen comprises a peptide having the amino acid sequence SLLMWITQV (SEQ ID NO:1).

16. The method of claim 12, wherein the NY-ESO-1 antigen comprises a cysteine terminated NY-ESO-1 peptide with an intervening flexible linker.

17. The method of claim 16, wherein the NY-ESO-1 antigen comprises a peptide having the amino acid sequence SLLMWITQV-LSPG-C(SEQ ID NO:5)

18. The method of claim 12, wherein the NY-ESO-1-expressing cancer is selected from a triple negative breast cancer, melanoma, myeloma and ovarian cancer.

19. The method of claim 12, wherein the composition further comprises a pharmaceutically acceptable carrier.

20. The method of claim 12, wherein the therapeutically effective amount of the anti-cancer composition is the amount effective to enhance uptake and activation of antigen presenting cells and promote a potent CD8+ T cell response in the subject.


SEQUENCE LISTING (5 sequences)

SEQ ID NO: 1 - protein, 9 aa, Artificial Sequence
  Synthetic Construct
SLLMWITQV

SEQ ID NO: 2 - protein, 11 aa, Artificial Sequence
  Synthetic Construct
SLLMWITQCFL

SEQ ID NO: 3 - protein, 9 aa, Artificial Sequence
  Synthetic Construct
SLLMWITQC

SEQ ID NO: 4 - protein, 9 aa, Artificial Sequence
  Synthetic Construct
QLSLLMWIT

SEQ ID NO: 5 - protein, 14 aa, Artificial Sequence
  Synthetic Construct
SLLMWITQVLSPGC